(19) United States
(12) Patent Application Publication
(10) Pub. No.: US 2025/0003967 A1
(43) Pub. Date: Jan. 2, 2025

(54) QUANTIFICATION OF AIRBORNE SARS-COV-2 USING A PAPER MICROFLUIDIC CHIP

(71) Applicant: ARIZONA BOARD OF REGENTS ON BEHALF OF THE UNIVERSITY OF ARIZONA, Tucson, AZ (US)

(72) Inventor: Jeong-Yeol Yoon, Tucson, AZ (US)

(21) Appl. No.: 18/711,553

(22) PCT Filed: Nov. 18, 2022
(86) PCT No.: PCT/US22/50506

§ 371 Date: May 17, 2024

Related U.S. Application Data
(60) Provisional application No. 63/281,605, filed on Nov. 19, 2021.

(51) Int. Cl. G01N 33/569; G01N 33/543; G01N 33/58; G01N 33/68

(52) U.S. Cl. CPC G01N 33/56983; G01N 33/54386; G01N 33/582; G01N 33/585; G01N 33/6854; G01N 2333/165

(57) ABSTRACT

The present disclosure provides kits and methods for detecting and/or quantifying coronavirus from aerosols and droplets.

### CROSS REFERENCE TO RELATED APPLICATIONS

This application claims the benefit of U.S. Provisional Application No. 63/281,605, filed on Nov. 19, 2021, entitled Direct Capture and Smartphone Quantification of Airborne SARS-CoV-2 on a Paper Microfluidic Chip. The entirety of the foregoing is hereby incorporated by reference.

### FIELD OF THE INVENTION

The field of the invention relates generally the measurement and/or detection of viruses.

### BACKGROUND

Severe acute respiratory syndrome coronavirus (SARS-CoV) was first identified in 2003 and spread into Southeast Asia and China. Roughly a decade later, another variant called Middle East respiratory syndrome coronavirus (MERS-CoV) has emerged. Another decade later, we have witnessed the worldwide pandemic of SARS-CoV-2, causing the coronavirus disease 2019 (COVID-19). It is expected to originate from bats, although the cause of its appearance has not been identified (Zhou et al., 2020).

SARS-CoV-2 infection may be mild or asymptomatic for most healthy people. Individuals with pre-existing conditions, such as obesity, diabetes, cancer, heart and lung disease, can be seriously ill, requiring intensive care units (ICUs), and may die with SARS-CoV-2. In addition, such morbidity and mortality can also occur in healthy individuals (Fozouni et al., 2020). Millions have been impacted by the COVID-19 pandemic.

COVID-19 diagnostics have typically been carried out on the nasopharyngeal swabs and, recently, on the nasal swabs from the infected patients (Zenhausem et al., 2021). Since SARS-CoV-2 spreads through the air, like SARS-CoV and MERS-CoV, it may be too late to contain its spread by testing the infected individuals. Therefore, the best approach would be direct detection from the air.

Bioaerosol refers to particulate matter with life characteristics suspended in the air (Hinds, 1982). Bioaerosol sizes range from 0.02 to 30 μm. In SARS-CoV and SARS-CoV-2, water droplets are generated from the human mouth through exhaling, coughing, and sneezing. SARS-CoV-2, with a diameter of 0.1 μm, has been believed to spread through the droplets >5 μm (thus “droplets” but not “aerosols”). Therefore, 6-feet or 2-m physical distancing has emerged as a safety precaution (WHO, 2021). However, several reports have been made that SARS-CoV-2 can spread with droplets smaller than 5 μm (i.e., aerosols) (Vuorinen et al., 2020; Chia et al., 2020; Hwang et al., 2021; Chen et al., 2020).

While the best practice to prevent SARS-CoV-2 infection is a face mask (Zhang et al., 2020), it is imperfect in capturing all bioaerosols, especially when SARS-CoV-2 spreads through smaller droplets and aerosols. In addition, some people cannot wear face masks due to medical conditions, and some others are not willing to wear face masks. Therefore, the best practice might be direct detection from the air.

The number of viruses in such air samples tends to be very low, which cannot easily be detected conventionally. Air samplers are necessary to increase the number of captured viruses.

There are three widely adopted methods for collecting viruses (or bacteria) from the air: impaction, impinging, and filtration (Terzieva et al., 1996; Fronczek and Yoon, 2015). The impaction method is the most common, where aerosols are aerodynamically separated by size according to their diameter. In the impinging method, the air is sucked in and passed through a device containing a liquid buffer to collect viruses or bacteria. The filtration method is economical and straightforward, where bioaerosols are collected using a fibrous or membrane filter. An additional incubation for more than 24 hours may be required for all three methods due to the small number of viruses and bacteria in the air.

Very few papers have been published to detect the COVID-19 aerosols from the air directly. In the small number of such published works, droplets or aerosols were collected using the traditional air samplers for a long time. Additionally, conventional laboratory analyses were used, such as reverse transcription-polymerase chain reaction (RT-PCR) and enzyme-linked immunosorbent assay (ELISA) (Piri et al., 2021; Su et al., 2020). These methods are not easy to be implemented in the field and cannot rapidly provide results.

In certain aspects, the invention encompasses a handheld, rapid, low-cost, smartphone-based paper microfluidic assay capable of directly detecting SARS-CoV-2 in the droplets or aerosols from the air, without needing an air sampler and the long collection time.

There is a strong need to develop a handheld, rapid, and extremely sensitive assay of coronaviruses such as SARS-CoV-2 amid the recent COVID-19 pandemic. The current gold standard is qRT-PCR from nasopharyngeal (NP) swabs, which requires laboratory and skilled personnel, and is time-consuming. Another emerging method is immunoassay from NP and other (nasal or buccal) swabs, including laboratory-based ELISA and field-based sandwich immunoassay strips (also known as rapid kits). As the immunoassays are not sensitive and have high limit of detection (high LOD), early-stage infections cannot be identified; SARS-CoV-2 is notoriously known for its early-stage propagation. In addition, they also suffer from false-positive results when used with complicated samples (NP or other swabs).

As such, there is a need to develop a device and a method for detecting and/or quantifying virus from aerosols and droplets. The present invention satisfies this need.

This background information is provided for the purpose of making information believed by the applicant to be of possible relevance to the present invention. No admission is necessarily intended, nor should it be construed, that any of the preceding information constitutes prior art against the present invention.

### BRIEF DESCRIPTION OF THE FIGURES

FIG. 1. Assay procedure. (1a) Chamber design with the dimensions and placements of a paper microfluidic chip and a sprayer. (1b-1e) The assay procedure, starting from the air collection, the addition of antibody-particle suspension, smartphone-based fluorescence microscopic imaging, and image processing with the MATLAB script.

FIG. 2. A smartphone-based fluorescence microscope and processed images. (2a) The light path diagram of a smartphone-based fluorescence microscope. (2b and 2c) 15 Photographs of a smartphone-based fluorescence microscope.

FIG. 3. Fluorescence images of a paper microfluidic channel for 0 ng/mL (control) and 600 pg/mL (sample) UV-inactivated SARS-CoV-2 in 10% saliva. Three different images from a single channel were taken with a benchtop fluorescence microscope.

FIG. 4. Assay results with a benchtop fluorescence microscope. Pixel counts from three different images of a single channel were summed and used as a single data point. Experiments were repeated four times (each time with three images), each using a different paper microfluidic chip. Average pixel counts (n=4) are plotted with the error bars representing standard errors, each time with different spraying/capture and different paper microfluidic chips. (4a) With two-time spraying, and (4b) with five-time spraying. * denotes p≤0.05, **p≤0.01, and n.s.=not significant.

FIG. 5. Assay results with a smartphone-based fluorescence microscope. Pixel counts from five different images of a single channel were summed and used as a single data point. Experiments were repeated four times (each time with five images), each using a different paper microfluidic chip. Average pixel counts (n=4) are plotted with the error bars representing standard errors, (5a) with two-time spraying and (5b) with five-time spraying. * denotes p≤0.05; ** denotes p≤0.01; n.s. denotes not significant.

FIG. 6. Standard curve to demonstrate the quantification capability of this droplet/aerosol assay. Pixel counts are plotted against the virus stock concentration in a sprayer. The distance of the paper microfluidic chip was fixed at 6 inches, and the number of sprays was fixed to two times. n=4; * denotes p≤0.05; ** denotes p≤0.01, in comparison with 0 pg/mL data.

FIG. 7. Droplets and aerosols were circulated through the chamber using two fans. Without fans, droplets and aerosols are sprayed generally from right to left, where the larger droplets mainly were found along the right-to-left linear path. With fans, droplets and aerosols are circulated in one direction, where the larger droplets can be found across the entire chamber. (Larger droplets can be identified from the bottom images, circled in yellow.)

FIG. 8. Assay results with a smartphone-based fluorescence microscope with two-time spraying and fans installed. All other conditions are identical to those shown in FIG. 5. n=4 each time with different spraying/capture and different paper microfluidic chips; ** denotes p≤0.01.

FIG. 9. Photographs of the chamber.

FIG. 10. Particle aggregation assays on paper microfluidic chips using monoclonal and polyclonal antibodies.

FIG. 11. Photographs of paper microfluidic chips after two-times and five-times spraying. Liquids are falling off the chip with five-times spraying.

FIGS. 12A-B. Raw images from a benchtop fluorescence microscope. Three different areas of a single channel were imaged for each assay. Paper microfluidic chip was placed at a 6-inch distance.

FIG. 13A-B. Raw images from a smartphone-based fluorescence microscope. Five different areas of a single channel were imaged for each assay. Paper microfluidic chip was placed at a 6-inch distance.

FIG. 14A-D. Raw images from a smartphone-based fluorescence microscope. Five different areas of a single channel were imaged for each assay.

FIG. 15. An example of a foldable smartphone-based fluorescence microscope, whose dimension is 10 cm×5 cm×15 cm.

FIG. 16. Cost analysis of a smartphone-based fluorescence microscope (all in US dollars).

### DESCRIPTION

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a 10% variation from the nominal value. It is to be understood that such a variation is always included in any given value provided herein, whether or not it is specifically referred to.

The inventors designed a system that simulates a human cough using a simple sprayer in a controlled chamber. Droplets or aerosols are passively collected on paper microfluidic chips without any collector, pump, fan, or filter. Antibody-conjugated submicron fluorescent particle suspension is added to the paper microfluidic chip, inducing antibody-antigen binding and subsequent particle aggregation. A low-cost smartphone-based fluorescence microscope was fabricated, used to quantify the extent of this particle aggregation from the microscopic images (Chung et al., 2021), and confirmed the presence of SARS-CoV-2 from the air. The device and method can slow the spread of SARS-CoV-2 and other emerging respiratory viruses.

One aspect of the invention pertains to a method for detecting and/or quantifying coronavirus from aerosols and droplets, said method comprises (a) adding an anti-virus antibody conjugated fluorescent particle suspension to a paper microfluidic chip comprising coronavirus particles and (b) quantifying said coronavirus particles.

Another aspect of the invention pertains a kit for detecting an airborne virus, said kit comprises a paper microfluidic chip with a pore size of about 5 μm to 15 μm depending on the type of sample suspension and a suspension of antibody conjugated fluorescent particles, and optionally a smartphone-based fluorescence microscope.

A further aspect of the invention pertains to a kit for detecting airborne coronavirus, said kit comprises (a) a device for detecting and/or quantifying airborne coronavirus comprising a paper microfluidic chip, comprising one or more microfluidic channels, wherein said paper has a pore size of about 5 μm to 15 μm, and wherein said channels have a width about 2 mm to about 5 mm and/or a channel length between channel length of about 20 mm to about 50 mm and (b) one or more reagents (e.g., fluorescent particles conjugated with different antibodies) for carrying out detection and/or quantification of one or more coronaviruses.

A further aspect of the invention pertains to a method for detecting an airborne virus, said method comprises (a) fabricating a paper microfluidic chip with multiple channels on it for simultaneously conducting multiple assays; (b) conjugating an antibody to fluorescent particles to obtain an anti-virus conjugated fluorescent submicron particle suspension; (c) passively collecting aerosols or droplets on a paper microfluidic chip (e.g., by placing the chip in the environment where aerosols and droplets are generated and circulated); (d) adding said antibody conjugated fluorescent particle suspension to the paper microfluidic chip; (e) allowing the particles to aggregate and facilitating imaging of individual particles (e.g. by allowing particles and viruses spread spontaneously throughout the paper microfluidic channel via capillary action); (f) imaging the aggregation of antibody conjugated fluorescent particles; (g) removing the background noises and autofluorescence from paper substrate using an optimized threshold intensity and isolating only the fluorescent particles; (h) binarizing an entire image; (i) removing the smaller size of particles to isolate only the aggregated particles; and ( ) relating the total pixel area to the virus concentration to construct a standard curve and estimate the virus concentration from an unknown sample.

A further aspect of the invention pertains to a method for detecting and/or quantifying coronavirus from aerosols and droplets, said method comprises (a) collecting aerosols and/or droplets that contain coronavirus on a paper microfluidic chip; (b) adding an anti-virus antibody conjugated fluorescent particle suspension to the paper microfluidic chip; and (c) imaging the individual particles (e.g., by allowing particles and viruses spread spontaneously throughout the paper microfluidic channel via capillary action and allowing the particles to aggregate to facilitate imaging of individual particles).

In some embodiments, a method for detecting and/or quantifying coronavirus from aerosols and droplets, said method comprises (a) adding an anti-virus antibody conjugated fluorescent particle suspension to a paper microfluidic chip comprising coronavirus particles; (b) quantifying said coronavirus particles. In some embodiments, the virus may be present in a concentration ranging from 10⁰ to 10⁵ virions.

In some embodiments, said paper microfluidic chip comprises nitrocellulose paper, cellulose paper, or polymeric fiber filter. In some embodiments, said aerosols and/or droplets comprising coronavirus particles are passively collected. For instance, said aerosols and/or droplets comprising coronavirus particles may be passively collected using neither aerosol samplers (filter sampler, liquid sampler, impinger sampler, etc.) nor purification, concentration, and amplification. In further embodiments, said method involves a single virus copy level detection of said virus. In some embodiments, said virus is a SARS-CoV-2 virus.

In some embodiments, said step (b) comprises imaging and counting aggregation of antibody-conjugated, fluorescent submicron particles is on said paper chip. In some embodiments, said step (b) comprising quantifying said coronavirus particles using a smartphone-based fluorescence microscope. In further embodiments, the volume of the antibody conjugated fluorescent particles is from 2 uL to 6 uL and wherein said suspension has a concentration from about 0.001% to about 0.04%. In further embodiments, said antibody is a polyclonal antibody or a monoclonal antibody. In some embodiments, the fluorescent particle is a fluorescent polystyrene particle.

In some embodiments, the method further comprises (1) fabricating a paper microfluidic chip with multiple channels on it (e.g., for simultaneously conducting multiple assays) and (2) conjugating an antibody to fluorescent particles to obtain an anti-virus antibody conjugated fluorescent submicron particle suspension, wherein said steps are performed prior to said steps (a)-(b).

In some embodiments, said method further comprises (i) imaging the aggregation of antibody conjugated fluorescent particles; (ii) removing the background noises and autofluorescence from paper substrate using an optimized threshold intensity and isolating only the fluorescent particles; (iii) binarizing an entire image; (iv) removing the smaller size of particles to isolate only the aggregated particles; and (v) relating the total pixel area to the virus concentration to construct a standard curve and estimate the virus concentration from an unknown sample, wherein said steps are performed after said steps (a)-(c).

In some embodiments, a method for detecting an airborne virus, said method comprises (a) fabricating a paper microfluidic chip with multiple channels on it for simultaneously conducting multiple assays; (b) conjugating an antibody to fluorescent particles to obtain an anti-virus conjugated fluorescent submicron particle suspension; (c) collecting (e.g., passively) aerosols or droplets on a paper microfluidic chip (e.g., by placing the chip in the environment where aerosols and droplets are generated and circulated); (d) adding said antibody conjugated fluorescent particle suspension to the paper microfluidic chip; (e) allowing the particles to aggregate and facilitating imaging of individual particles (e.g. by allowing particles and viruses spread spontaneously throughout the paper microfluidic channel via capillary action); (f) imaging the aggregation of antibody conjugated fluorescent particles; (g) removing the background noises and autofluorescence from paper substrate using an optimized threshold intensity and isolating only the fluorescent particles; (h) binarizing an entire image; (i) removing the smaller size of particles to isolate only the aggregated particles; and (j) relating the total pixel area to the virus concentration to construct a standard curve and estimate the virus concentration from an unknown sample.

In some embodiments, a method for detecting and/or quantifying coronavirus from aerosols and droplets, said method comprises (a) collecting aerosols and/or droplets that contain coronavirus on a paper microfluidic chip; (b) adding an anti-virus antibody conjugated fluorescent particle suspension to the paper microfluidic chip; (c) imaging the individual particles (e.g., by allowing particles and viruses spread spontaneously throughout the paper microfluidic channel via capillary action and allowing the particles to aggregate to facilitate imaging of individual particles).

In some embodiments, a kit for detecting an airborne virus, said kit comprises a paper microfluidic chip with a pore size of about 5 μm to 15 μm depending on the type of sample suspension, a suspension of antibody conjugated fluorescent particles, and optionally a smartphone-based fluorescence microscope.

In some embodiments, said kit further comprises a smartphone-based fluorescence microscope comprising a microscope attachment, a light source (such as LED), a battery to power said light source (such as LED) (e.g., a button battery), and an optical filter. In further embodiments, a microscope attachment, an LED, a battery to power LED (e.g., a button battery), and an optical filter are housed within a plastic enclosure to block ambient lighting. In some embodiments, said microscope attachment comprises a bandpass filter or acrylic films (also known as “filter cards”).

In some embodiments, a kit for detecting airborne coronavirus, said kit comprises (a) a device for detecting and/or quantifying airborne coronavirus comprising a paper microfluidic chip, comprising one or more microfluidic channels, wherein said paper has a pore size of about 5 μm to 15 μm, and wherein said channels have a width about 2 mm to about 5 mm and/or a channel length between channel length of about 20 mm to about 50 mm; and (b) one or more reagents (e.g., fluorescent particles conjugated with different antibodies) for carrying out detection and/or quantification of one or more coronaviruses.

In some embodiments, said kit further comprises a smartphone-based fluorescence microscope comprising a microscope attachment, a light source (such as LED), a battery to power said light source (such as LED) (e.g., a button battery), and an optical filter. In further embodiments, a microscope attachment, an LED, a battery to power LED (e.g., a button battery), and an optical filter are housed within a plastic enclosure to block ambient lighting. In some embodiments, said microscope attachment comprises a bandpass filter or acrylic films (also known as “filter cards”).

### EXAMPLES

### Materials and Methods

### 2.1. SARS-CoV-2 Samples

SARS-CoV-2 Isolate USA-WA1/2020, was deposited by Dr. Natalie J. Thomburg at the U.S. Centers for Disease Control Prevention (CDC) and obtained from the World Reference Center for Emerging Viruses and Arboviruses (WRCEVA). SARS-CoV-2 was passaged on mycoplasma negative Vero cells (ATCC #CCL-81) at an MOI of 0.005 for 48 h. Supernatant and cell lysate were combined, subjected to a single freeze-thaw, and then centrifuged at 3000 RPM for 10 min to remove cell debris. Concentration ranged from 10⁶ PFU/mL to 10⁷ PFU/mL, corresponding to 2 to 6 ng/mL, respectively. Virus stock was poured into a 15-cm petri dish and irradiated in a Bio-Rad GS Gene Linker UV Chamber on the ‘sterilize’ setting twice for 90 s with a brief swirl in between. The virus's complete inactivation was confirmed by standard plaque assay or 50% tissue culture infectious dose (TCID₅₀). All live virus manipulations were performed in a biosafety level 3 laboratory, and procedures were approved by the University of Arizona's Institutional Biosafety Committee.

### 2.2. Antibody Conjugation to Fluorescent Polystyrene Particles

Yellow-green fluorescent carboxylated polystyrene particles (CAYF500NM; Magsphere, Inc., Pasadena, CA, USA) were used for assaying SARS-CoV-2. According to the manufacturer, their diameter was 500 nm, the excitation peak was 488 nm (blue), and the emission peak was 509 nm. Rabbit polyclonal antibody to SARS-CoV-2 (40588-T30; SinoBiological, Inc., Wayne, PA, USA) was covalently conjugated to the particles via carbodiimide reaction. Detailed information and optimizations can be found in the previous works (Chung et al., 2019; Chung et al., 2021; Kim et al., 2021; Park and Yoon, 2015). The final particle concentration was 0.06 μg/μL, optimized for showing the maximum extent of particle aggregation (Chung et al., 2021; Kim et al., 2021). Particle stocks were stored in a refrigerator.

### 2.3. Paper Microfluidic Chip

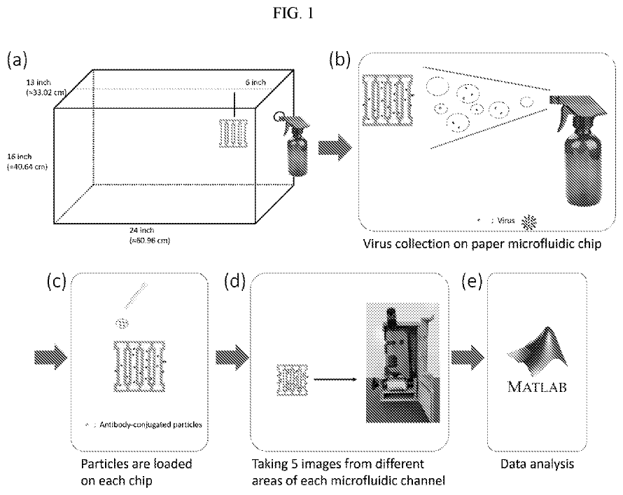

The paper microfluidic chip was designed, optimized, and wax-printed in the same way used in the previous work (Kim et al., 2021; Chung et al., 2021). There were four channels in each chip. Each channel was 21 mm long and 2.4 wide, with dumbbell-shaped squares at both ends (FIG. 1). Nitrocellulose paper was used (CN95; Sartorius, Göttingen, Germany). Capillary flow rate was 65-115 s over 40 mm as reported by the manufacturer. Thickness is 240-270 μm.

### 2.4. Air Chamber

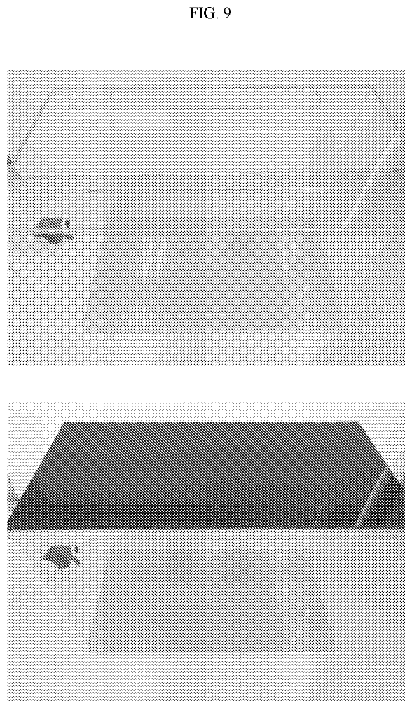

A fish tank was purchased and used as an air chamber (Fish Tanks Direct, North Venice, FL, USA). A hole on one side was drilled, where a sprayer (Amber Spray Bottles; Maredash, Shenzhen, China) was inserted and sealed. FIG. 1A showed the schematics and dimensions of this air chamber. A photograph of this chamber was shown in FIG. 9. The chamber dimension (e.g., 24-inch length) roughly corresponded to the one-tenth scale of a typical classroom, as demonstrated in the previous work (Kwon et al., 2014). Following the Research Laboratory and Safety Services requirements at the University of Arizona, the chamber was inspected, approved for this research, placed within a chemical hood, sprayed with ethanol, thoroughly wiped before and after all experiments. Air collection experiments were performed at the biosafety level 2 laboratory.

2.5. Direct air sampling on paper microfluidic chips and assay procedure UV-inactivated SARS-CoV-2 was spiked into the 10% v/v human saliva solutions (from human donors, confirmed negative for SARS-CoV-2) with varying concentrations and loaded to the sprayer. The final concentration was 600 pg/mL, comparable to that in human saliva from active COVID-19 patients (Zhu et al., 2020). Concentrations of 0, 200, 400, and 800 pg/mL were additionally used to demonstrate the quantification capability. A paper microfluidic chip was placed 6 in (15.2 cm; one-quarter point of the chamber length), 12 in (30.5 cm; one-half point), or 24 in (61 cm; full length) away from the sprayer nozzle. It was sprayed two times manually (simulating typical human coughs), consuming 3 mL, as well as five times (mimicking repetitive human coughs) consuming 7.5 mL. Spraying made the paper chips wet. After 15 minutes, the paper microfluidic chips were collected from the chamber. The chips were incubated at room temperature for additional 10 minutes, allowing liquid to be evaporated. 4 μL of antibody conjugated particle suspension was then pipetted to the center of each channel and incubated for another 5 minutes allowing the particles to interact with the viruses. The paper microfluidic chip was then imaged and analyzed with a smartphone-based fluorescence microscope. This procedure was illustrated in FIG. 1B through IE.

### 2.6. Smartphone-Based Fluorescence Microscopic Imaging of Paper Microfluidic Chips

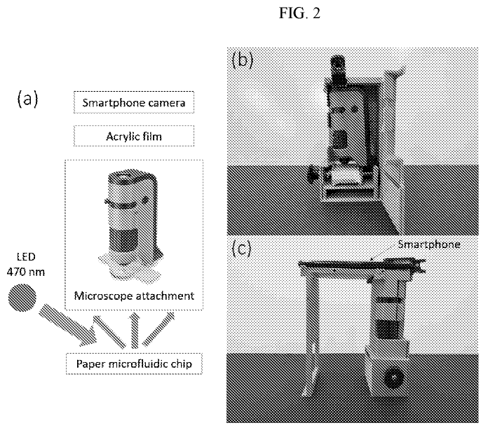

A smartphone-based fluorescence microscope was designed and built following the method described in Chung et al. (Chung et al., 2021), with the modifications described below. A commercial microscope attachment to a smartphone was purchased and used (MicroFlip 100-250× High Power Pocket Microscope; Carson Optical, NY, USA). A 460 nm LED (WP7113QBC/G; Digi-Key Electronics, MN, USA) was used as a light source for the excitation of fluorescent particles. The excitation wavelength was slightly shorter than the peak excitation of the particles (488 nm) to avoid the overlap with the emission signal while providing sufficient excitation to the particles, as confirmed with the fluorescence images. A 9-V battery powered this LED. Acrylic films (#382 and #15; Color Filter Booklet; Edmund Optics, AZ, USA) were used as the low-cost excitation and emission filters placed between the microscope attachment and smartphone camera. A smartphone camera (Samsung Galaxy S20 FE 5G; Samsung Electronics America, Inc., NJ, USA) was used to image each paper channel and isolate only the aggregated particles, which would be discussed in the following section. All components (a microscope attachment, an LED, a 9-V battery, acrylic filter, and a smartphone) were mounted on a foldable stand and stage designed in SolidWorks and 3D-printed using Creality Ender-3 (Shenzhen Creality 3D Technology Co. Ltd.; Shenzhen, Guangdong, China) with PETG filament (Overture; Wilmington, DE, USA). This smartphone-based fluorescence microscope was illustrated in FIG. 2. For the comparison purpose, a benchtop fluorescence microscope was also used to image the paper chips, whose procedure could be found in the previous work (Kim et al., 2021).

### 2.7. Image Processing

The MATLAB (Me MathWorks, Inc.; Natick, MA, USA) script used in this study was based on the previous work (Chung et al., 2019; Kim et al., 2021). Fluorescence images were acquired from five different locations of each channel since the field-of-view (FOV) was small and could not represent the overall particle aggregation behavior over the length of the channel. Each image was split into three color channels (red, green, and blue). The green channel images were used to capture the green fluorescence (peaked at 509 nm) of the particles. The color histogram of the fluorescence signal was investigated to optimize the intensity threshold value (Park et al., 2014). The pixels with intensities lower than 60 (out of 255) were eliminated to remove background noise (McCracken et al., 2016), which was optimized through a series of experiments using only the fluorescent particles on the paper. (For the benchtop fluorescence microscope analysis, the threshold value was 200). The images were then binarized (black and white). Next, size thresholding was applied to isolate only the aggregated particles. Only the spheres with 10 to 400 pixels (the same size thresholding was considered and used for the benchtop fluorescence microscope analysis); all other spheres were considered noise or artifacts (especially dust).

### 2.8. Air Sampling on Paper Microfluidic Chips Under Forced Fan Circulation

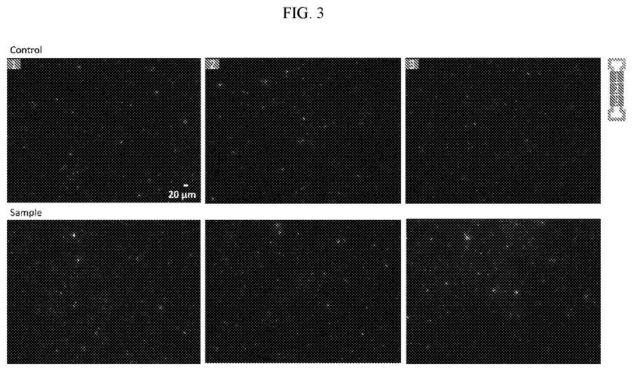

Two computer case cooling fans (NFD1293259B-2F; Y.S. Tech., Garden Grove, CA, USA, and FD08025, Masscool, Walnut, CA, USA) were installed in the chamber, as shown in FIG. 3. A simple voltage divider circuit controlled the speed of fans and, subsequently, circulation rate. Two fans were installed to circulate the air in one circular direction. All other conditions were the same as those described in section 2.5.

### 3. RESULTS AND DISCUSSION

3.1. Confirmation of Particle Aggregation with SARS-CoV-2

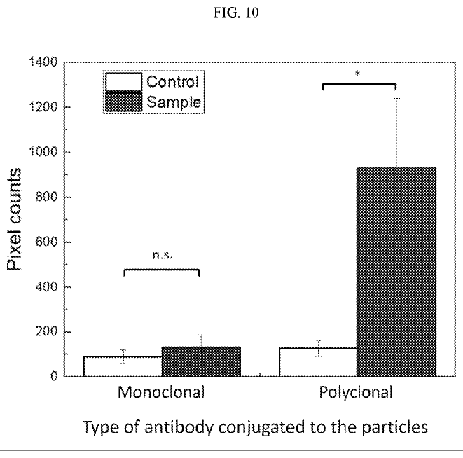

Two types of antibodies were used to assay SARS-CoV-2 from the air samples. One was rabbit monoclonal antibody, and the other was rabbit polyclonal antibody, both to SARS-CoV-2. Experiments were initially conducted by sequentially pipette-adding the SARS-CoV-2 solution (0 and 600 pg/mL in 10% saliva) and the antibody-particle suspension on each paper microfluidic channel to demonstrate the initial feasibility. A benchtop fluorescence microscope was used for this initial investigation, and the results were shown in FIG. 3. A substantial number of particle aggregations were found from the fluorescence microscopic images of the paper microfluidic channels, demonstrating the capability of identifying SARS-CoV-2 using this method. (Me same experiments using monoclonal antibody conjugated particles as well as rabbit monoclonal anti-SARS-CoV-2, catalog number 40588-R0004 from SinoBiological, Inc. It was found that the extent of particle aggregation was much inferior to that with a polyclonal antibody. Refer to FIG. 10)

3.2. Droplet/Aerosol Assay with a Benchtop Fluorescence Microscope

Droplets and aerosols containing SARS-CoV-2 were generated from a sprayer and passively collided with the paper microfluidic chips, as shown in FIG. 1. The SARS-CoV-2 concentration of 600 pg/mL (or 600 fg/μL) was determined from the typical SARS-CoV-2 concentration found among COVID-19 infected patients (Zhu et al., 2020). The amount of SARS-CoV-2 colliding to the paper microfluidic chip should be much lower than that inside the sprayer, and the fraction settling within a single channel would be even lower than that. The control was the 10% saliva without any SARS-CoV-2. Only the pixels representing aggregated particles were summed using the MATLAB script from the fluorescence microscope images.

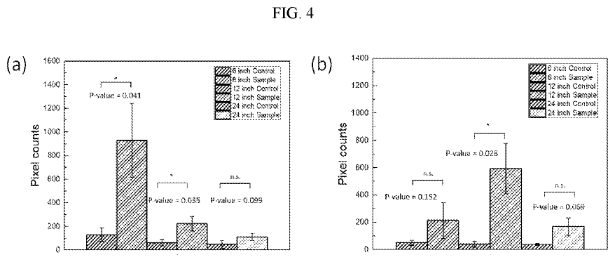

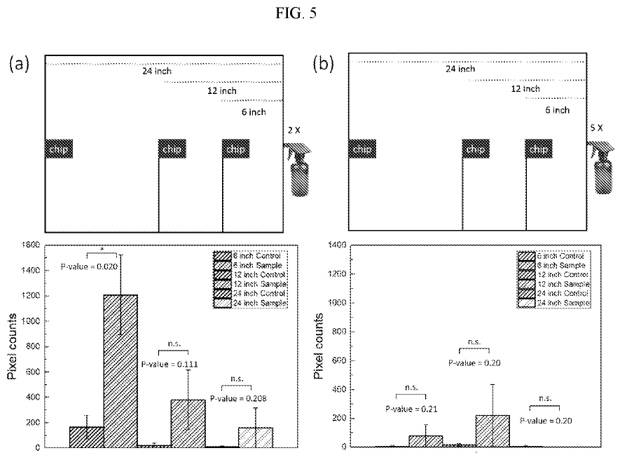

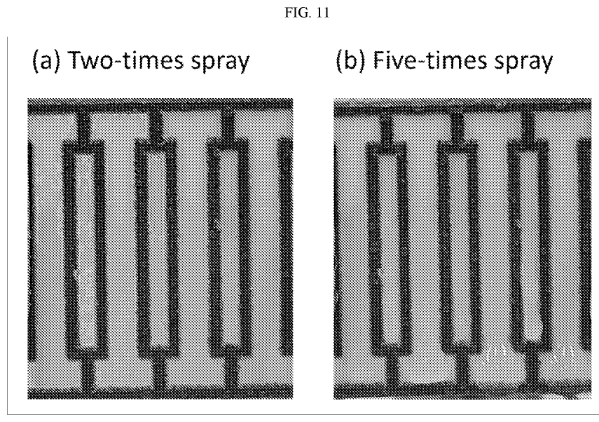

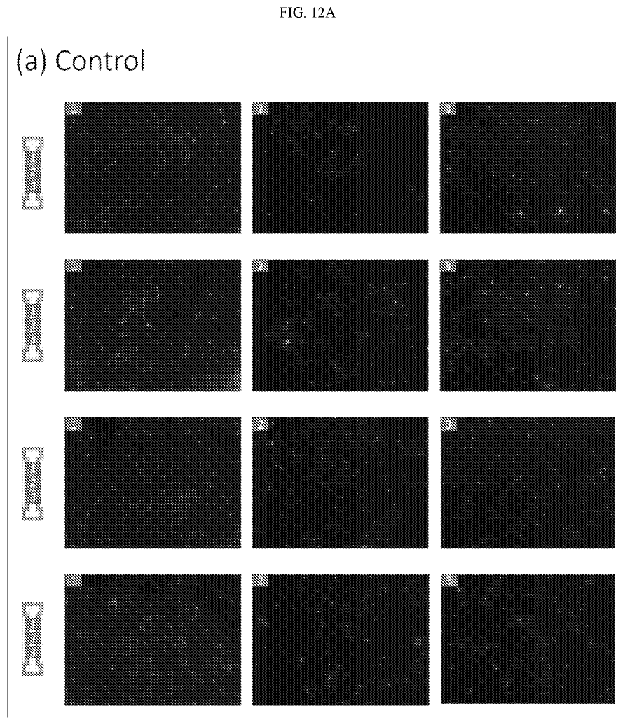

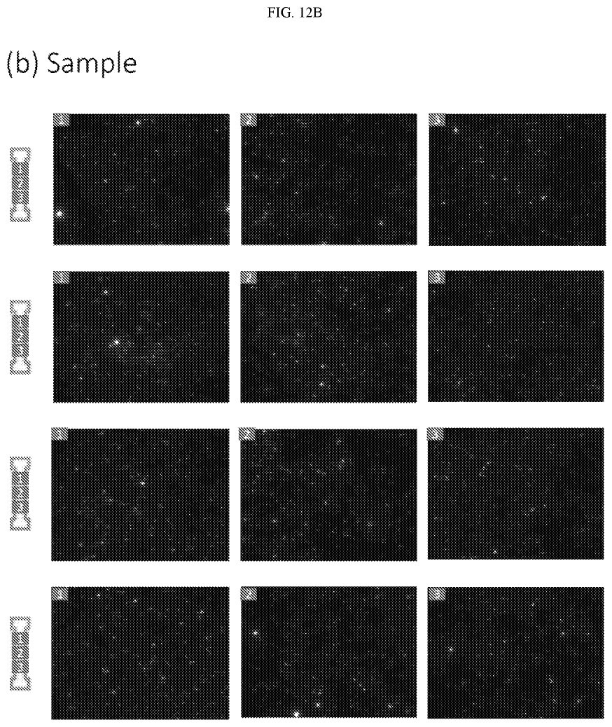

It was first investigated whether the sensor system works with a benchtop fluorescence microscope. FIG. 4A showed the pixel counts of aggregated particles on the paper microfluidic chip, where the SARS-CoV-2 samples were sprayed at the distances of 6, 12, and 24 inches (15.2, 30.5, and 60 cm, respectively), corresponding to the quarter, half, and full length of the air chamber (FIG. 1A). Polyclonal antibody-conjugated particles were used in all further experiments. Total pixel counts decreased as the distance increased from 6 to 24 inches, indicating that the number of captured droplets/aerosols in each channel decreased with the distance. Means and standard errors were collected from four different experiments, each time using different paper microfluidic chips. The results with 6 and 12 inches were significantly different from those of the negative controls (0 pg/mL 10% saliva) (p<0.05). FIG. 4B showed the result with five-time spraying. Higher pixel counts were recorded at 12 and 24 inches than those with two-time spraying, improving the results. However, the pixel counted at 6 inches were significantly lower than those of 12 inches. They were also lower than those with two-time spraying at 6 inches. Liquids were falling off from the paper microfluidic chip, as shown in FIG. 11, leading to the loss of liquid and subsequently inefficient capturing. All raw images were summarized in FIG. 12.

3.3. Droplet/Aerosol Assay with a Smartphone-Based Fluorescence Microscope

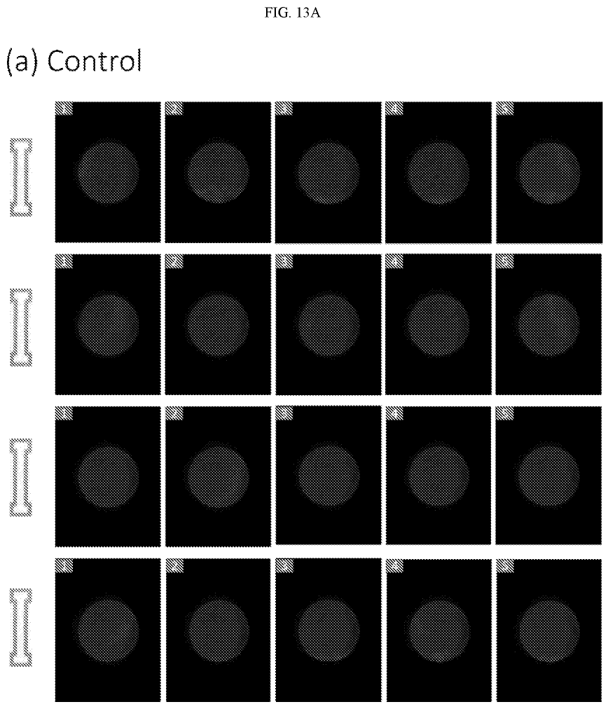

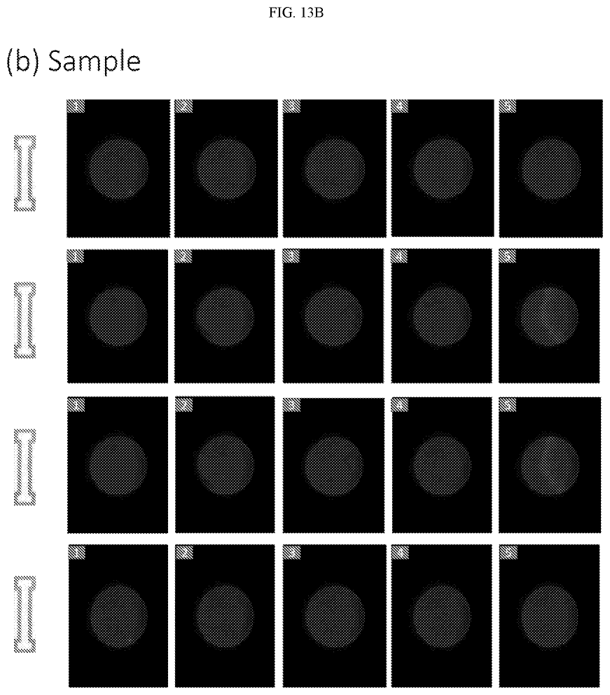

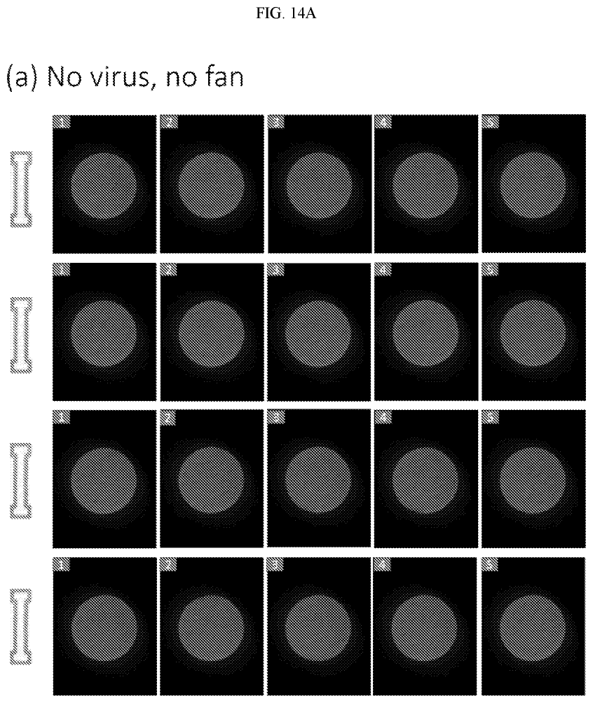

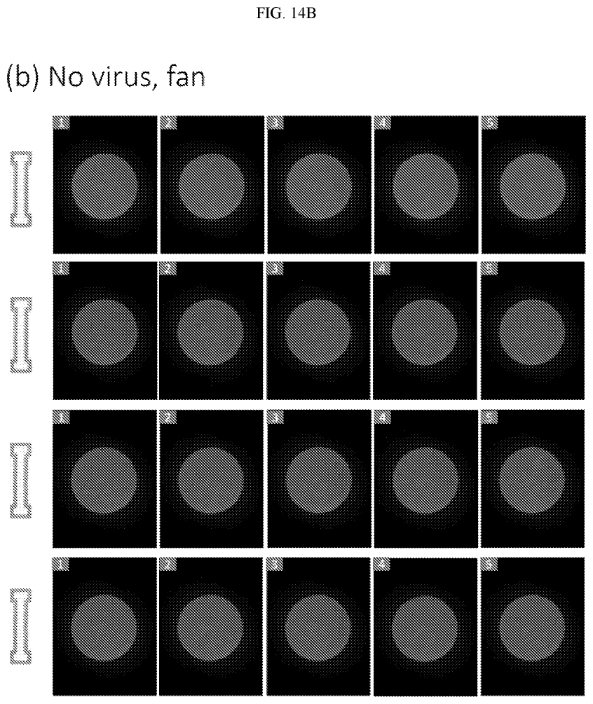

Experiments were repeated using a smartphone-based fluorescence microscope (FIG. 2). Five images were collected from a single channel since the FOV of a smartphone microscope was smaller than that of a benchtop fluorescence microscope. FIG. 5 showed the results. Overall, the pixel counts were lower than those with a benchtop fluorescence microscope. However, the trend was identical: the pixel counts decreased as the distance increased, and the five-time spraying at a 6-inch distance did not work. The sample data was significantly different from that of the control group at a 6-inch distance. All raw images used in FIG. 5 were summarized in FIG. 13.

### 3.4. Quantification Capability of Smartphone-Based Droplet/Aerosol Assay

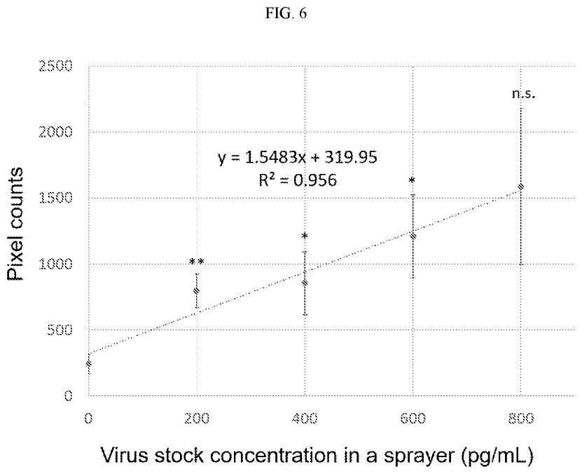

To demonstrate the quantification capability, additional experiments were conducted with varying concentrations (0, 200, 400, 600, and 800 pg/mL) with two-time spraying at a 6-inch distance. The result was shown in FIG. 6, showing satisfactory linearity (R²=0.956). Most data points were significantly different (p<0.05) from that of 0 pg/mL except for 800 pg/mL, although its p-value of 0.053 was still close to 0.05. Using this linear regression equation, the sample assay results were converted and shown in FIG. 5A to the concentration: 570, 40, and 0 pg/mL, respectively. Considering 600 pg/mL concentration, the paper microfluidic chips at 12-inch and 24-inch distances captured 7% and 0% of the same at 6-inch distance, respectively.

### 3.5. Smartphone-Based Droplet/Aerosol Assay Under Forced Air Circulation

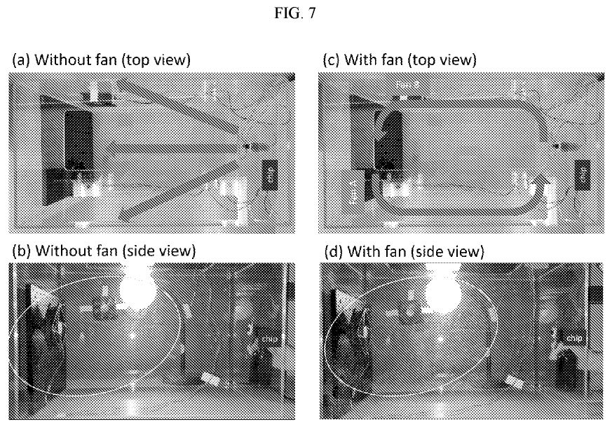

Additional experiments were performed to see if this method could be applied to the environment that can better represent the air-conditioned rooms. As shown in FIG. 7, two fans were installed inside the chamber, designed to circulate the air. A paper chip was installed on the same side of the sprayer. Larger droplets can be visually identified using a bright light bulb, as shown in FIG. 7D. Most larger droplets traveled from the right-side sprayer to the other direction at least 24 inches, indicating that larger droplets must travel for a minimum of 48 inches to reach the paper chip. Smaller droplets and aerosols can travel farther than 48 inches, although they cannot be identified from the photographs.

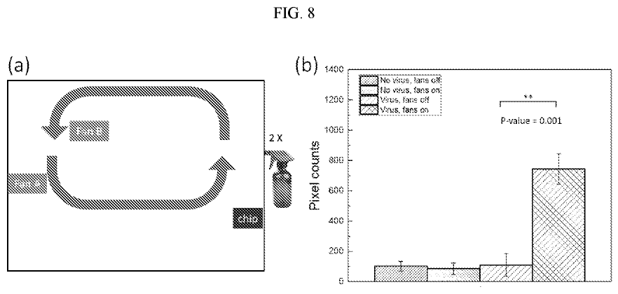

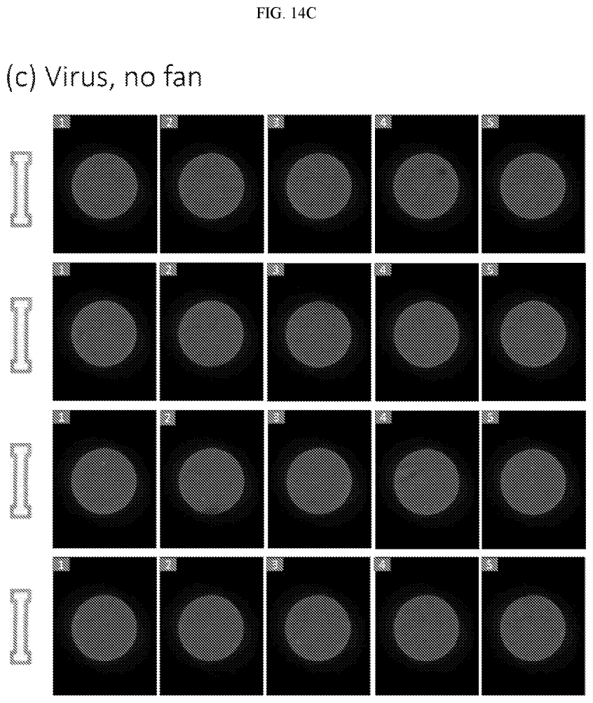

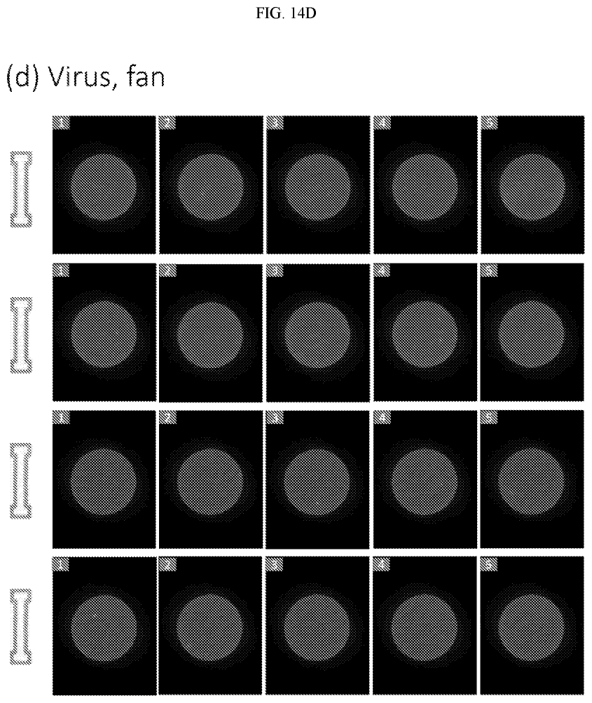

The assay results were summarized in FIG. 8. With viruses and fans off, the pixel counts were not significantly different from those with no virus (i.e., deionized water) and fans off (first column) and fans on (second column). Comparison of third and fourth columns (fans off vs. on, both with viruses) indicated that viruses could not be captured on the paper chip with no air circulation. Comparison of second and fourth columns (with vs. without viruses, both with fans on) indicated that the fans without viruses could not generate positive signals, i.e., no false positives. The average pixel counts with viruses and fans (fourth column) were 740±100, showing a significant difference from the other three (p≤0.01). This number was smaller than the 6-inch sample data shown in FIG. 5A but higher than the 12-inch and 24-inch sample data, indicating the role of air circulation towards effective virus capture on paper chips. All raw images used in FIG. 8 were summarized in FIG. 14.

### 4. CONCLUSION

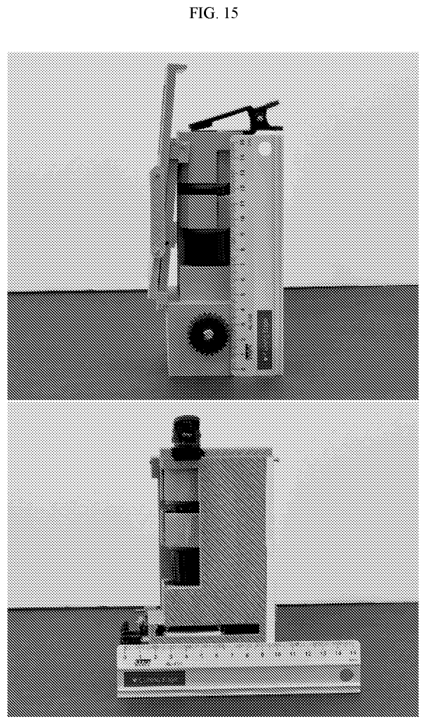

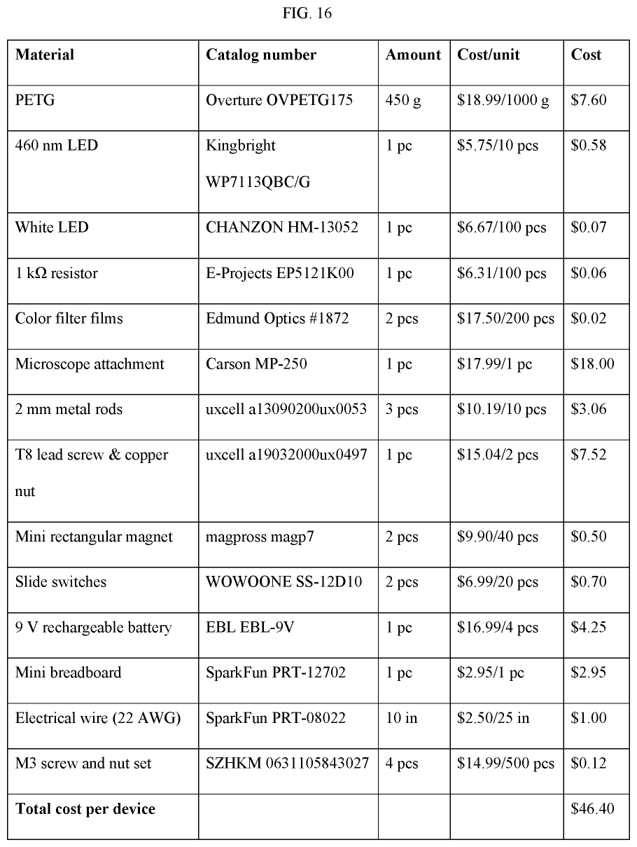

The SARS-CoV-2 from the sprayed droplets/aerosols directly on a paper microfluidic chip was collected without using any sampler device. Neither pumps nor fans were necessary (besides the fans used to simulate air conditioning) to collect these droplets, and the collection was entirely passive. Assays were conducted directly on the paper microfluidic chip without the need for sample collection, transfer, dilution, and purification. Antibody conjugated particle suspension was pipette-added to the channel, allowing particle aggregation induced by antibody-virus binding. A smartphone-based fluorescence microscope captured fluorescent images, and a MATLAB script isolated and quantified particle aggregation. Despite the small number of viruses captured on each channel, they could be detected with only two sprays (presumably equivalent to two coughs) at a 6-inch (15.2 cm) distance. Successful capture and detection were also demonstrated with the air circulated through the chamber. The method required a paper microfluidic chip (neither pre-loading nor immobilization was necessary), antibody-particle suspension, and a smartphone-based fluorescence microscope (requiring low-cost and off-the-shelf components, such as an LED, a 9-V battery, acrylic film, and a microscope attachment. The total cost of parts and supplies for a smartphone-based fluorescence microscope was US$46.60 (excluding a smartphone), as shown in FIG. 16. It was also foldable and handheld, whose dimension is 10 cm×5 cm×10 cm, as shown in FIG. 15.

### Non-Limiting List of Embodiments

The following is list of exemplary embodiments of the invention:
1. A method for detecting and/or quantifying coronavirus from aerosols and droplets, said method comprising
(a) adding an anti-virus antibody conjugated fluorescent particle suspension to a paper microfluidic chip comprising coronavirus particles; and
(b) quantifying said coronavirus particles.
2. The method of embodiment 1, wherein the virus is present in a concentration ranging from 10⁰ to 10⁵ virions.
3. The method of embodiment 1, wherein said paper microfluidic chip comprises nitrocellulose paper, cellulose paper, or polymeric fiber filter. For instance, the paper chip may have more than one channels. The channels may be configured so that the paper chip can simultaneously conduct more than 1 assay.
4. The method of embodiment 1, wherein aerosols and/or droplets comprising coronavirus particles are passively collected. For instance, said aerosols and/or droplets comprising coronavirus particles are passively collected using neither aerosol samplers (filter sampler, liquid sampler, impinger sampler, etc.) nor purification, concentration, and amplification.
5. The method of embodiment 1, wherein said method involves a single virus copy level detection of said virus.
6. The method of embodiment 1, wherein said virus is a SARS-CoV-2 virus.
7. The method of embodiment 1, wherein said step (b) comprises imaging and counting aggregation of antibody-conjugated, fluorescent submicron particles on said paper chip.
8. The method of embodiment 1, wherein said step (b) comprising quantifying said coronavirus particles using a smartphone-based fluorescence microscope.
9. A kit for detecting an airborne virus comprising
a paper microfluidic chip with a pore size of about 5 μm to 15 μm depending on the type of sample suspension,
a suspension of antibody conjugated fluorescent particles, and
optionally a smartphone-based fluorescence microscope.
10. The method of embodiment 1, wherein the volume of the antibody conjugated fluorescent particles is from about 2 uL to about 6 uL (preferably about 2-4 uL or 4-6 uL or about 4 uL or about 6 uL) and wherein said suspension has a concentration from about 0.001% to about 0.04% (preferably about 0.001-0.01% or about 0.004-0.008% or about 0.006%).
11. A kit for detecting airborne coronavirus, said kit comprising
(a) a device for detecting and/or quantifying airborne coronavirus comprising a paper microfluidic chip, comprising one or more microfluidic channels, wherein said paper has a pore size of about 5 μm to 15 μm, and wherein said channels have a width about 2 mm to about 5 mm and/or a channel length between channel length of about 20 mm to about 50 mm; and
(b) one or more reagents (e.g., fluorescent particles conjugated with different antibodies) for carrying out detection and/or quantification of one or more coronaviruses.
12. The method of embodiment 1, wherein said antibody is a polyclonal antibody or a monoclonal antibody.
13. The method of embodiment 1, wherein the fluorescent particle is a fluorescent polystyrene particle.
14. The method of embodiment 1, wherein said method further comprises:
(1) fabricating a paper microfluidic chip with one or more channels on it (e.g., for more than one channels (preferably 3-8 channels for simultaneously conducting multiple assays);
(2) conjugating an antibody to fluorescent particles to obtain an anti-virus antibody conjugated fluorescent submicron particle suspension;
wherein said steps are performed prior to said step (a).
15. The method of embodiment 1, wherein step (b) comprises:
(i) imaging the aggregation of antibody conjugated fluorescent particles;
(ii) removing the background noises and autofluorescence from paper substrate using an optimized threshold intensity and isolating only the fluorescent particles;
(iii) binarizing an entire image;
(iv) removing the smaller size of particles to isolate only the aggregated particles;
(v) relating the total pixel area to the virus concentration to construct a standard curve and estimate the virus concentration from an unknown sample.
16. A method for detecting an airborne virus, said method comprising:
(a) fabricating a paper microfluidic chip with multiple channels on it for simultaneously conducting multiple assays;
(b) conjugating an antibody to fluorescent particles to obtain an anti-virus conjugated fluorescent submicron particle suspension;
(c) collecting (e.g., passively) aerosols or droplets on a paper microfluidic chip (e.g., by placing the chip in the environment where aerosols and droplets are generated and circulated);
(d) adding said antibody conjugated fluorescent particle suspension to the paper microfluidic chip;
(e) allowing the particles to aggregate and facilitating imaging of individual particles (e.g. by allowing particles and viruses spread spontaneously throughout the paper microfluidic channel via capillary action);
(f) imaging the aggregation of antibody conjugated fluorescent particles;
(g) removing the background noises and autofluorescence from paper substrate using an optimized threshold intensity and isolating only the fluorescent particles;
(h) binarizing an entire image;
(i) removing the smaller size of particles to isolate only the aggregated particles;
(j) relating the total pixel area to the virus concentration to construct a standard curve and estimate the virus concentration from an unknown sample.
17. The kit of embodiment 9 or embodiment 11, wherein said device further comprises a smartphone-based fluorescence microscope comprising a microscope attachment, a light source (such as LED), a battery to power said light source (such as LED) (e.g., a button battery), and an optical filter.
18. The kit of embodiment 9 or embodiment 11, wherein a microscope attachment, an LED, a battery to power LED (e.g., a button battery), and an optical filter are housed within a plastic enclosure to block ambient lighting.
19. The kit of embodiment 9 or embodiment 11, wherein said microscope attachment comprises a bandpass filter or acrylic films (also known as “filter cards”).
20. A method for detecting and/or quantifying coronavirus from aerosols and droplets, said method comprising
(a) collecting aerosols and/or droplets that contain coronavirus on a paper microfluidic chip;
(b) adding an anti-virus antibody conjugated fluorescent particle suspension to the paper microfluidic chip;
(c) imaging the individual particles (e.g., by allowing particles and viruses spread spontaneously throughout the paper microfluidic channel via capillary action and allowing the particles to aggregate to facilitate imaging of individual particles).

### REFERENCES

Chen W, Zhang N, Wei J, Yen H L, Li Y, 2020. Build. Environ. 176: 106859. https://doi.org/10.1016/j.buildenv.2020.106859.
Chia P Y, Coleman K K, Tan Y K, Ong S W X, Gum M, Lau S K, Lim X F, Lim A S, Sutjipto S, Lee P H, Son T T, Young B E, Milton D K, Gray G C, Schuster S, Barkham T, De P P, Vasoo S, Chan M, Ang B S P, Tan B H, Leo Y S, Ng O T, Wong M S Y, Marimuthu K. 2020. Nat. Commun. 2020, 11: 2800. https://doi.org/10.1038/s41467-020-16670-2.
Chung S, Breshears L E, Perea S, Morrison C M, Betancourt W Q, Reynolds K A, Yoon, J Y, 2019. ACS Omega 4: 11180-11188. https://doi.org/10.1021/acsomega.9b00772
Chung S, Breshears L E, Gonzales A, Jennings C M, Morrison C M, Betancourt W Q, Reynolds K A, Yoon J Y, 2021. Nat. Protoc. 16: 1452-1475. https://doi.org/10.1038/s41596-020-00460-7
Fozouni P, Son S, Diaz de León Derby M, Knott G J, Gray C N, D'Ambrosio M V, Zhao C, Switz N A, Kumar G R, Stephens S I, Boehm D, Tsou C L, Shu J, Bhuiya A, Armstrong M, Harris A R, Chen P Y, Osterloh J M, Meyer-Franke A, Joehnk B, Walcott K, Sil A, Langelier C, Pollard K S, Crawford E D, Puschnik A S, Phelps M, Kistler A, DeRisi J L, Doudna J A, Fletcher D A, Ott M, 2020. Cell 184: 1-11. https://doi.org/10.1016/j.cell.2020.12.001
Fronczek C F, Yoon J Y, 2015. J. Lab. Automat. 20: 390-410. https://doi.org/10.1177/2211068215580935
Hinds, W. C., 1982. Aerosol technology: properties, behaviour, and measurement of airborne particles. John Wiley & Sons: Hoboken.
Hwang S E, Chang J H, Oh B, Heo J, 2021. Int. J. Infect. Dis. 104: 73-76. https://doi.org/10.1016/j.ijid.2020.12.035
Kim S., Romero-Lozano, A, Hwang D S, Yoon J Y, 2021. J. Hazard. Mater. 413: 125338. https://doi.org/10.1016/j.jhazmat.2021.125338
Kwon H J, Fronczek C F, Angus S V, Nicolini A M, Yoon J Y, 2014. J. Lab. Autom. 19: 322-331. https://doi.org/10.1177/2211068213504205
McCracken K E, Angus S V, Reynolds K A, Yoon J Y, 2016. Sci. Rep. 6: 27529. https://doi.org/10.1038/srep27529
Park T S, Baynes C, Cho S I, Yoon J Y, 2014. RSC Adv. 4: 24356-24362. https://doi.org/10.1039/c4ra01471e
Park T S, Yoon J Y, 2015. IEEE Sens. J. 15: 1902-1907. https://doi.org/10.1109/JSEN.2014.2367039
Piri A, Kim, HR, Park D H, Hwang J, 2021. J. Hazard. Mater. 413: 125417. https://doi.org/10.1016/j.jhazmat.2021.125417
Su X, Sutarlie L, Loh X J, 2020. Chem. An Asian J. 15: 4241-4255. https://doi.org/10.1002/asia.202001051
Terzieva S, Donnelly J, Ulevicius V, Grinshpun S A, Willeke K, Stelma G N, Brenner K P, 1996. Appl. Environ. Microbiol. 62: 2264-2272. https://doi.org/10.1128/aem.62.7.2264-2272.1996
Vuorinen V, Aamio M, Alava M, Alopaeus V, Atanasova N, Auvinen M, Balasubramanian N, Bordbar H, Erästö P, Grande R, Hayward N, Hellsten A, Hostikka S, Hokkanen J, Kaario O, Karvinen A, Kivistö I, Korhonen M, Kosonen R, Kuusela J, Lestinen S, Laurila E, Nieminen H J, Peltonen P, Pokki J, Puisto A, Råback P, Salmenjoki H, Sironen T, Österberg M, 2020. Saf. Sci. 130, 104866, https://doi.org/10.1016/j.ssci.2020.104866.
World Health Organization (WHO). Modes of transmission of virus causing COVID-19: implications for IPC precaution recommendations https:/www.who.int/news-room/commentaries/detail/modes-of-transmission-of-virus-causing-covid-19-implications-for-ipc-precaution-recommendations (accessed Apr. 16, 2021)
Zenhausem R, Chen C H, Yoon J Y, 2021. Biomicrofluidics 15: 011503. https://doi.org/10.1063/5.0041089
Zhang R, Li Y, Zhang A L, Wang Y, Molina M J, 2020. Proc. Natl. Acad. Sci. U.S.A. 117: 14857-14863. https://doi.org/10.1073/pnas.2009637117
Zhou P, Yang X L, Wang X G, Hu B, Zhang L, Zhang W, Si H R, Zhu Y, Li B, Huang C L, Xiao G F, Shi Z L, 2020. Nature 579: 270-273. https://doi.org/10.1038/s41586-020-2012-7
Zhu J, Guo J, Xu Y, Chen X, 2020. J. Infect. 81: e48-e50. https://doi.org/10.1016/j.jinf.2020.06.059

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.

1. A method for detecting and/or quantifying coronavirus from aerosols and droplets, said method comprising
(a) adding an anti-virus antibody conjugated fluorescent particle suspension to a paper microfluidic chip comprising coronavirus particles; and
(b) quantifying said coronavirus particles.

2. The method of claim 1, wherein the virus is present in a concentration ranging from 10⁰ to 10⁵ virions.

3. The method of claim 1, wherein said paper microfluidic chip comprises nitrocellulose paper, cellulose paper, or polymeric fiber filter.

4. The method of claim 1, wherein aerosols and/or droplets comprising coronavirus particles are passively collected.

5. The method of claim 1, wherein said method involves a single virus copy level detection of said virus.

6. The method of claim 1, wherein said virus is a SARS-CoV-2 virus.

7. The method of claim 1, wherein said step (b) comprises imaging and counting aggregation of antibody-conjugated, fluorescent submicron particles on said paper chip.

8. The method of claim 1, wherein said step (b) comprising quantifying said coronavirus particles using a smartphone-based fluorescence microscope.

9. (canceled)

10. The method of claim 1, wherein the volume of the antibody conjugated fluorescent particles is from about 2 uL to about 6 uL and wherein said suspension has a concentration from about 0.001% to about 0.04%.

11. (canceled)

12. The method of claim 1, wherein said antibody is a polyclonal antibody or a monoclonal antibody.

13. The method of claim 1, wherein the fluorescent particle is a fluorescent polystyrene particle.

14. The method of claim 1, wherein said method further comprises:
(1) fabricating a paper microfluidic chip with one or more channels on it;
(2) conjugating an antibody to fluorescent particles to obtain an anti-virus antibody conjugated fluorescent submicron particle suspension;
wherein said steps are performed prior to said step (a).

15. The method of claim 1, wherein step (b) comprises:
(i) imaging the aggregation of antibody conjugated fluorescent particles;
(ii) removing the background noises and autofluorescence from paper substrate using an optimized threshold intensity and isolating only the fluorescent particles;
(iii) binarizing an entire image;
(iv) removing the smaller size of particles to isolate only the aggregated particles;
(v) relating the total pixel area to the virus concentration to construct a standard curve and estimate the virus concentration from an unknown sample.

16. A method for detecting an airborne virus, said method comprising:
(a) fabricating a paper microfluidic chip with multiple channels on it for simultaneously conducting multiple assays;
(b) conjugating an antibody to fluorescent particles to obtain an anti-virus conjugated fluorescent submicron particle suspension;
(c) collecting aerosols or droplets on a paper microfluidic chip;
(d) adding said antibody conjugated fluorescent particle suspension to the paper microfluidic chip;
(e) allowing the particles to aggregate and facilitating imaging of individual particles;
(f) imaging the aggregation of antibody conjugated fluorescent particles;
(g) removing the background noises and autofluorescence from paper substrate using an optimized threshold intensity and isolating only the fluorescent particles;
(h) binarizing an entire image;
(i) removing the smaller size of particles to isolate only the aggregated particles; and
(j) relating the total pixel area to the virus concentration to construct a standard curve and estimate the virus concentration from an unknown sample.

17.-19. (canceled)

20. A method for detecting and/or quantifying coronavirus from aerosols and droplets, said method comprising
(a) collecting aerosols and/or droplets that contain coronavirus on a paper microfluidic chip;
(b) adding an anti-virus antibody conjugated fluorescent particle suspension to the paper microfluidic chip; and
(c) imaging the individual particles.

21. The method of claim 16, wherein step c) comprises placing the chip in the environment where aerosols and droplets are generated and circulated.

22. The method of claim 16, wherein step e) comprises allowing particles and viruses to spread spontaneously throughout the paper microfluidic channel via capillary action.

23. The method of claim 20, wherein step c) comprises allowing particles and viruses to spread spontaneously throughout the paper microfluidic channel via capillary action and allowing the particles to aggregate to facilitate imaging of individual particles.
